(19) United States
(12) Patent Application Publication
(10) Pub. No.: US 2022/0125338 A1
(43) Pub. Date: Apr. 28, 2022

(54) ABNORMALITY DETERMINATION APPARATUS, METHOD, AND COMPUTER-READABLE MEDIUM

(71) Applicant: NEC Corporation, Minato-ku, Tokyo (JP)

(72) Inventors: Yuji OHNO, Tokyo (JP); Masahiro KUBO, Tokyo (JP); Toshinori HOSOI, Tokyo (JP)

(73) Assignee: NEC Corporation, Minato-ku, Tokyo (JP)

(21) Appl. No.: 17/430,848

(22) PCT Filed: Feb. 18, 2019
(86) PCT No.: PCT/JP2019/005757

§ 371 Date: Aug. 13, 2021

(51) Int. Cl. A61B 5/11; A61B 5/00; G08B 21/02

(52) U.S. Cl. CPC A61B 5/1118; A61B 5/702; A61B 5/1128; G08B 21/02; A61B 5/4809; A61B 5/7267; A61B 5/0077

(57) ABSTRACT

The abnormality determination apparatus includes a movement quantity detection unit and an abnormality level determination unit. The movement quantity detection unit detects movement quantities of a plurality of parts of the monitored person. Restraint information is information indicating a restraint status of the monitored person. The abnormality level determination unit determines an abnormality level of the monitored person on the basis of the movement quantities of the plurality of parts detected by the movement quantity detection unit and the restraint information.

### TECHNICAL FIELD

The present invention relates to an abnormality determination apparatus, method, and computer-readable medium and, specifically, relates to an abnormality determination apparatus, method, and computer-readable medium that determine whether there is an abnormality in a monitored person such as a patient.

### BACKGROUND ART

Hospitalized patients who are in a hospital or the like, for example, include patients who are likely to have behavioral problems such as falling from a bed, pulling out a tube, uttering a strange sound, and using violence. Patients having behavioral problems are often in a state of “restlessness” or “delirium”. Some healthcare workers such as nurses and care workers spend 20 to 30 percent of their time to deal with hospitalized patients who are likely to have behavioral problems, which causes strain on time for healthcare workers to focus their efforts on doing their care work.

Patent Literature 1 discloses a monitoring system that monitors patients and detects the delirium of patients. In the monitoring system described in Patent Literature 1, an analysis unit detects motion events of patients from image data of patients obtained over time. An evaluation unit classifies the detected motion events into delirium typical motion events and non-delirium typical motion events. A delirium determination unit determines a delirium score indicating the probability of delirium of a patient from the duration, strength, type, place and/or occurrence of a delirium typical motion event.

### CITATION LIST

### Patent Literature

Patent Literature 1: Japanese Unexamined Patent Application Publication No. 2014-528314

### SUMMARY OF INVENTION

### Technical Problem

In some cases, a patient who is in an acute hospital or the like is put into physical restraint in order to prevent problems such as getting injured due to a fall or pulling out an infusion needle or to prevent a disease from getting worse. Physical restraint includes mitten restraint, trunk restraint, upper limb restraint, lower limb restraint, four-limb restraint and the like, for example. In the case where a patient is restrained, since the motion of a restrained part is restricted, a correlation between time variation of movement (motion) and delirium is likely to change from the case where physical restraint is not used. However, physical restraint of a patient is not taken into consideration in Patent Literature 1. Therefore, in Patent Literature 1, there is a possibility that the probability of delirium cannot be determined properly when a patient is restrained.

In view of the foregoing, an object of the present disclosure is to provide an abnormality determination apparatus, method, and computer-readable medium that can improve the accuracy of detecting an abnormal condition of a monitored person.

### Solution to Problem

To achieve the above object, the present disclosure provides an abnormality determination apparatus including a movement quantity detection means for detecting movement quantities of a plurality of parts of a monitored person, and an abnormality level determination means for determining an abnormality level of the monitored person on the basis of the movement quantities of the plurality of parts and restraint information indicating a restraint status of the monitored person.

The present disclosure also provides an abnormality determination method including detecting movement quantities of a plurality of parts of a monitored person, and determining an abnormality level of the monitored person on the basis of the movement quantities of the plurality of parts and restraint information indicating a restraint status of the monitored person.

The present disclosure also provides a computer readable medium storing a program causing a computer to execute a process including detecting movement quantities of a plurality of parts of a monitored person, and determining an abnormality level of the monitored person on the basis of the movement quantities of the plurality of parts and restraint information indicating a restraint status of the monitored person.

### Advantageous Effects of Invention

An abnormality determination apparatus, method, and computer-readable medium according to the present disclosure can improve the accuracy of detecting an abnormal condition of a monitored person.

### BRIEF DESCRIPTION OF DRAWINGS

FIG. 1 is a block diagram schematically showing an abnormality determination apparatus according to the present disclosure.

FIG. 2 is a block diagram showing an abnormality determination apparatus according to a first example embodiment of the present disclosure.

FIG. 3 is a schematic view showing a patient.

FIG. 4 is a graph showing the movement quantity of a certain part detected by a movement amount detection unit.

FIG. 5 is a graph showing a volume detected by a volume detection unit.

FIG. 6 is a schematic view showing calculation of an abnormality level.

FIG. 7 is a table showing the correspondence between restraint information and movement quantity factors.

FIG. 8 is a graph showing a restlessness level calculated by an abnormality level determination unit.

FIG. 9 is a flowchart showing an operation procedure in the abnormality determination apparatus.

FIG. 10 is a schematic view showing a patient lying on a bed.

FIG. 11 is a block diagram showing a biological information processing system.

FIG. 12 is a graph showing a specific example of a restlessness score.

FIG. 13 is a block diagram showing a system used for learning of an identification model.

FIG. 14 is a flowchart showing an operation procedure in the biological information processing system.

FIG. 15 is a graph showing a specific example of a restlessness score.

FIG. 16 is a block diagram showing a configuration example of an information processing apparatus that can be used as various apparatuses in the present disclosure.

### DESCRIPTION OF EMBODIMENTS

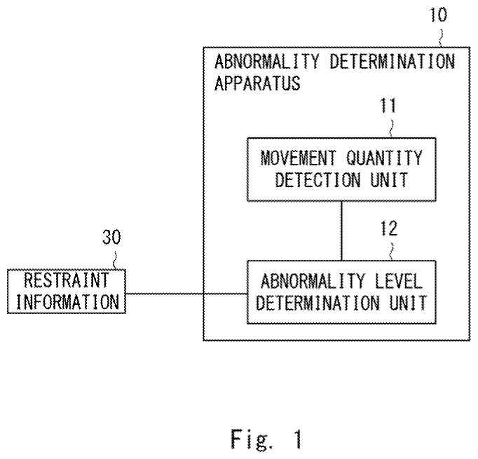

Prior to describing example embodiments of the present disclosure, the overview of the present disclosure will be described. FIG. 1 schematically shows an abnormality determination apparatus according to the present disclosure. An abnormality determination apparatus 10 includes a movement quantity detection means 11 and an abnormality level determination means 12.

The movement quantity detection means 11 detects the movement quantities of a plurality of parts of a monitored person such as a hospitalized patient. The movement quantity indicates the quantity related to the motion of a monitored person. The movement quantity may include at least one of the measure, the speed, and the acceleration of a motion, or the periodicity and the time variation of them, for example. The movement quantity detection means 11 detects the movement quantities of a plurality of parts by using image data obtained by capturing images of a monitored person, for example. Restraint information 30 is information indicating a restraint status of a monitored person. The abnormality level determination means 12 determines the level of abnormality of a monitored person on the basis of the movement quantities of a plurality of parts detected by the movement quantity detection means 11 and the restraint information 30.

In the present disclosure, the level of abnormality is determined on the basis of the movement quantities of a plurality of parts of a monitored person detected by the movement quantity detection means 11 and the restraint information 30 of the monitored person. The movement quantity of each part detected by the movement quantity detection means 11 can vary depending on whether physical restraint is used or not, and which part of the body is restrained when physical restraint is used. In the present disclosure, the level of abnormality is determined from the movement quantity by using the restraint information 30, and therefore the abnormal condition of a monitored person is detected properly depending on the restraint status, and the accuracy of detection of an abnormality is improved.

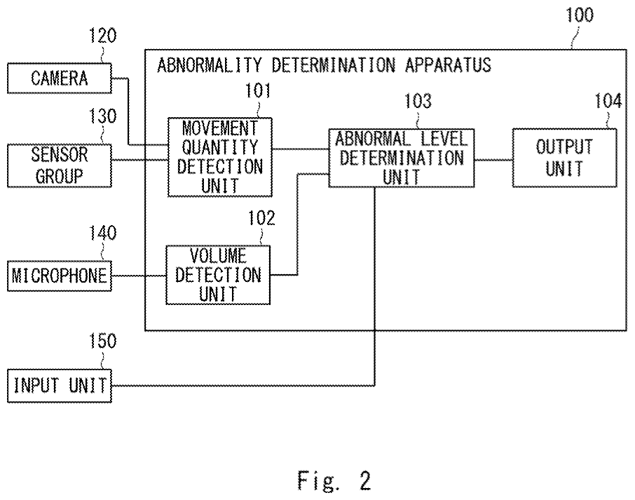

Example embodiments of the present disclosure will be described hereinafter in detail with reference to the drawings. FIG. 2 shows an abnormality determination apparatus according to a first example embodiment of the present disclosure. An abnormality determination apparatus 100 includes a movement amount detection unit 101, a volume detection unit 102, an abnormality level determination unit 103, and an output unit 104. The abnormality determination apparatus 100 is configured as a computer device that includes a memory and a processor, for example. The abnormality determination apparatus 100 is connected to a camera 120, a sensor group 130, a microphone 140, and an input unit 150. The abnormality determination apparatus 100 corresponds to the abnormality determination apparatus 10 in FIG. 1. The abnormality determination apparatus 100 is used to determine whether a monitored person such as a patient who is in a hospital or the like is in abnormal condition or not, for example.

According to the observation of the present inventors, it is found that, at least in the case of neurosurgical patients, they are likely to be in the state of restlessness (restless state) that their behavior is excessive and restless before actually taking problematic behavior. The “restless state” can include not only the state where the behavior is simply excessive and restless but also the state where a patient is not calm and unable to normally control their mental state. Since the restless state occurs due to at least one of physical pain or delirium, the term “restless state” includes delirium in this specification.

Specific behavior of patients in the restless state may include moving their arms and legs with no reason, having their bodies shaking, concentrating on a certain action in an unnatural manner, saying something logically unclear, not listening to what nurses or caregivers say and the like. The restless state may further include behavior not harmful to a patient, such as that related to their need to urinate, for example. In this example embodiment, the abnormality determination apparatus 100 determines the level of the restless state of a patient on the basis of the movement quantity of the patient.

The camera 120 outputs image data (video data) of a monitored person such as a patient to the abnormality determination apparatus 100. The camera 120 is placed above a bed on which a patient is laying, for example. The camera 120 captures an image of the patient lying on the bed, and outputs the captured image data to the abnormality determination apparatus 100. The microphone 140 outputs at least one of a voice uttered by a patient or a sound generated by the behavior of a patient to the abnormality determination apparatus 100. The camera 120 and the microphone 140 may be configured in the same device or may be configured as separate devices. The image data or the like may be stored separately in a storage device such as a semiconductor storage device or a magnetic storage device.

The sensor group 130 includes one or more sensors capable of detecting the motion of a patient. The sensor group 130 includes at least one of an acceleration sensor to be worn on a patient, an acceleration sensor to be placed on a bed or a bed sheet, or a radio sensor placed near a bed, for example. The sensor group 130 may include a sensor that measures the inner state of a patient. The inner state of a patient means the state of a patient which cannot be determined directly by another person from the outside, and it includes a mental state or the like, for example. The sensor that measures the inner state can include a sensor that measures biological information such as a heartbeat sensor and a temperature sensor, for example. The sensor group 130 may include a sensor for measuring the blood pressure, and a sensor for measuring the respiration rate of a patient.

The input unit 150 inputs information related to a patient to the abnormality determination apparatus 100. The input unit 150 includes an input device such as a keyboard and a mouse, for example. The information input by the input unit 150 includes restraint information. The restraint information is information indicating the restraint status of a patient. The restraint information contains information indicating which type of physical restraint is used on a patient, for example. Further, in the case where a certain type of physical restraint is used on a patient, the restraint information contains information about time when this type of physical restraint is performed. Furthermore, in the case where physical restraint on a patient is removed, the restraint information contains information about time when the physical restraint is removed. Note that “restraint” is also called “constraint”. The restraint information input from the input unit 150 corresponds to the restraint information 30 in FIG. 1. The information input from the input unit 150 may contain other information written in an electronic health record such as a result of hourly rounding at night and medication information, for example.

When there is an abnormality in a patient, a healthcare worker presses a button placed bedside or the like in some cases. The information input by the input unit 150 may contain information indicating that a healthcare worker has pressed a button. The button may be a physical button or an electronic button displayed on a touch panel of a tablet device. A healthcare worker may input information about the details of care given to a patient, such as talking to the patient or adjusting the bed, to the abnormality determination apparatus 100 by using the input unit 150. Further, the information input by the input unit 150 may contain button pressed information of a nurse call button pressed by a patient.

The movement amount detection unit 101 acquires image data and sensor data from the camera 120 and the sensor group 130, respectively. The movement amount detection unit 101 detects the movement quantity related to the motion of a patient on the basis of the acquired data. The movement amount detection unit 101 detects, as the movement quantity, at least one of the measure, the speed, and the acceleration of a motion, or the periodicity and the time variation of them, for example. The movement amount detection unit 101 detects the movement quantities of a plurality of parts of a patient. The movement amount detection unit 101 corresponds to the movement amount detection unit 11 in FIG. 1.

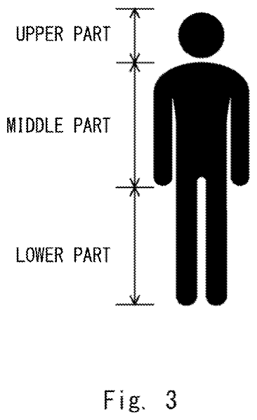

FIG. 3 shows a patient whose movement quantity is to be detected. In this example, it is assumed that a patient is lying on a bed. The movement amount detection unit 101 detects the movement quantity for each of the upper part, the middle part and the lower part of the patient, for example. The upper part corresponds to the head of the patient, the middle part corresponds to the upper body of the patient excluding the head, and the lower part corresponds to the lower body of the patient, for example. The movement amount detection unit 101 detects the movement quantity for each part of a patient such as the upper part, the middle part (upper limb), and the lower part (lower limb) by using the image data input from the camera 120, for example. The movement amount detection unit 101 may detect the movement quantity of a given part such as the hand, foot, head or the like of a patient from the sensor data obtained from the sensor group 130.

The movement amount detection unit 101 may standardize the movement quantity for each part in such a way that the maximum value of the movement quantity becomes a predetermined value, such as 1, for example. Further, the movement amount detection unit 101 may standardize the movement quantity in such a way that the maximum value of the movement quantity becomes 1 in each of the period before and after the type of physical restraint changes on the basis of the restraint information. To be specific, the movement amount detection unit 101 may standardize the movement quantity in such a way that the maximum value of the movement quantity becomes 1 in each of the period where the type of physical restraint is “no physical restraint” and the period where the type of physical restraint is “upper limb restraint”, for example.

The volume detection unit (volume detection means) 102 acquires the volume related to a sound uttered by a patient or a sound around a patient on the basis of audio data acquired from the microphone 140. The volume detection unit 102 detects at least one of the volume level, the frequency characteristics, or the periodicity and the time variation of them, for example. The volume detection unit 102 may standardize the volume in such a way that the maximum value of the volume becomes a predetermined value, such as 1, for example.

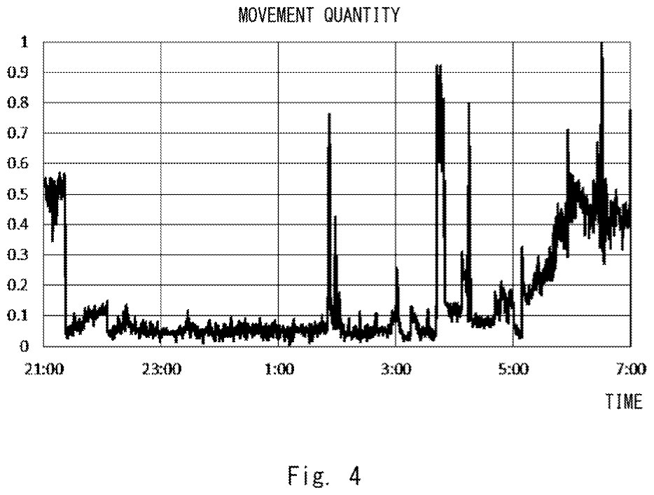

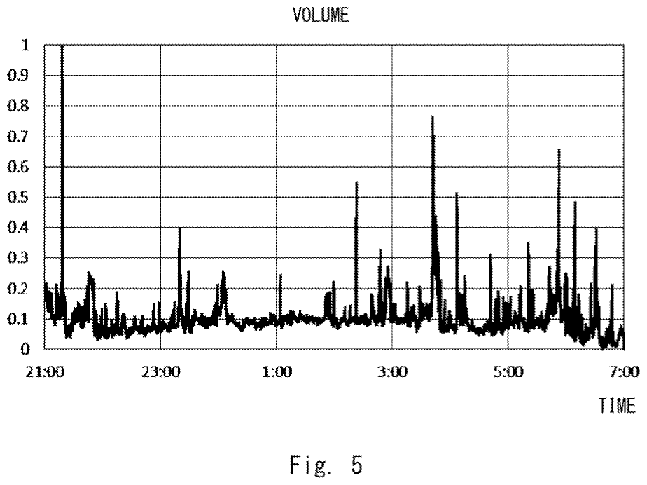

FIG. 4 shows the movement quantity of a certain part detected by the movement amount detection unit 101. FIG. 5 shows the volume detected by the volume detection unit 102. In FIG. 4, the horizontal axis indicates time, and the vertical axis indicates the standardized movement quantity. In FIG. 5, the horizontal axis indicates time, and the vertical axis indicates the standardized volume. The movement amount detection unit 101 detects how much a patient has moved in a predetermined time period every predetermined time interval such as 1 minute, for example. Further, the volume detection unit 102 detects the volume in a predetermined time period every predetermined time interval such as 1 minute, for example. The movement quantity detected by the movement amount detection unit 101 and the volume detected by the volume detection unit 102 can vary from moment to moment as shown in FIGS. 4 and 5.

The abnormality level determination unit 103 determines the level of abnormality (abnormality level) of a patient on the basis of the movement quantities of a plurality of parts detected by the movement amount detection unit 101, the volume detected by the volume detection unit 102, and the restraint information input by the input unit 150. The abnormality level includes abnormal, normal, and unclear, for example. “Abnormal” may be classified into a plurality of levels indicating the degree of abnormal behavior. To be specific, “abnormal” may be classified into “extremely abnormal”, “abnormal”, and “slightly abnormal”. Alternatively, “abnormal” may be classified into “abnormality level 1” to “abnormality level 5” and the like. The way of representing the abnormality level is not particularly limited as long as it represents the degree of abnormality. The abnormality level may be represented by a numerical value such as a score, for example. The abnormality level relates to the level of restless state (restlessness level) of a patient that can be determined on the basis of the motion of the patient. In this case, as the level is higher, the probability of taking problematic behavior is high, or the possibility of causing a severe problem is high. The abnormality level determination unit 103 corresponds to the abnormality level determination unit 12 in FIG. 1.

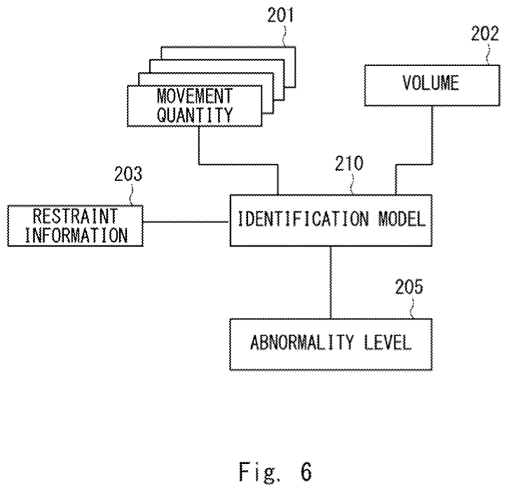

The abnormality level determination unit 103 determines (calculates) the abnormality level by using an identification model for calculating the abnormality level from the movement quantity and the volume, for example. FIG. 6 schematically shows calculation of the abnormality level. The abnormality level determination unit 103 inputs the movement quantities 201 of a plurality of parts and the volume 202 to an identification model 210. A linear model is used as the identification model 210, for example. The identification model, however, is not limited to a linear model, and a more complex model may be used.

For example, the abnormality level determination unit 103 calculates the abnormality level (restlessness level) L by using the following equation.

L=αuMu+αmMm+αlMl+βV (1)

In the above equation, Mu indicates the movement quantity of the upper part, Mm indicates the movement quantity of the middle part, Ml indicates the movement quantity of the lower part, and V indicates the volume. Further, au indicates the movement quantity factor of the upper part, am indicates the movement quantity factor of the middle part, αl indicates the movement quantity factor of the lower part, and β indicates the volume factor. In the above equation, αu, αm, αl, and β correspond to the identification model 210.

For example, when the movement quantity of a patient is small and the volume is small, the above-described identification model 210 outputs a value indicating “normal”. On the other hand, when the movement quantity is large and the volume is large, the identification model 210 outputs a value indicating “abnormal”. When there are many sudden motions or many repetitive actions, the identification model 210 may output a value indicating “abnormal”. Further, when there are many sudden utterances, the identification model 210 may output a value indicating “abnormal”. Even when the above case is applicable, if the action of a patient corresponds to a predetermined action registered in advance, the abnormality level determination unit 103 may set the value of the restlessness level to a value indicating “normal”. The predetermined action may be pressing a nurse call button and waiting for a nurse to come, and then going to a toilet, for example. Such action can be represented by the identification model as shown in the above equation 1.

The movement quantity of each part of a patient can vary depending on the restraint status of the patient. For example, when “upper limb restraint” is used on a patient, the movement quantity of the middle part, particularly, is expected to be reduced compared with when physical restraint is not used. In this example embodiment, the abnormality level determination unit 103 selects the identification model 210 to be used for the calculation of the restlessness level depending on the restraint information 203. To be specific, the abnormality level determination unit 103 changes the values of the above-described movement quantity factors αu, αm, and al, particularly, depending on the restraint information 203, and thereby changes the calculation of the restlessness level depending on the restraint status.

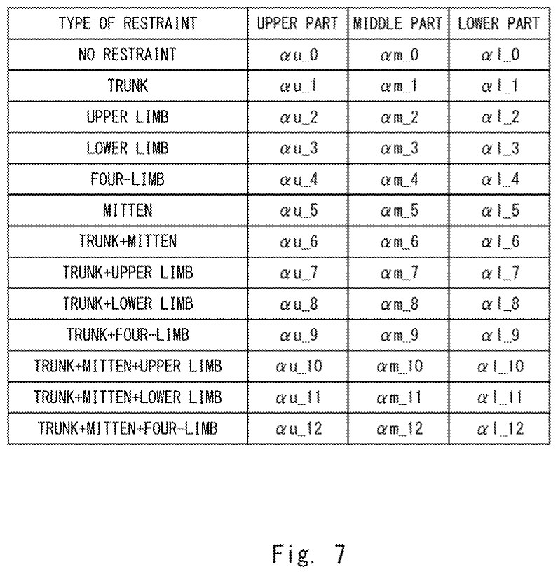

FIG. 7 shows the correspondence between the restraint information and the movement quantity factors αu, αm, and αl. αu_0 to αu_12, αm_0 to αm_12, and αl_0 to αl_12 shown in FIG. 7 represent any given real numbers. The abnormality level determination unit 103 checks which type of physical restraint is used on a patient by referring to the restraint information 203, and determines the values of the movement quantity factors αu, αm, and αl depending on the type of physical restraint by referring to the table shown in FIG. 7. When physical restraint is not used on a patient, for example, the abnormality level determination unit 103 sets the values of the movement quantity factors αu, αm, and αl to αu_0, αm_0, and αl_0, respectively. When “trunk restraint” and “mitten restraint” are used on a patient, the abnormality level determination unit 103 sets the values of the movement quantity factors αu, αm, and αl to αu_6, αm_6, and αl_6, respectively. In this manner, the values of the movement quantity factors αu, am, and αl are determined on the basis of the restraint information 203, so that the restlessness level is calculated using the identification model corresponding to the type of physical restraint.

The above-described identification model can be generated by performing machine learning using the movement quantity detected by the movement amount detection unit 101 and the volume detected by the volume detection unit 102 as explanatory variables, and using information (label) indicating whether it is abnormal or not which is input by a healthcare worker as an objective variable. For example, by preparing training data containing the detected movement quantity and volume and the label input by a healthcare worker through the input unit 150 or the like for each restraint status, and performing machine learning with use of this training data, the identification model corresponding to each restraint status can be generated.

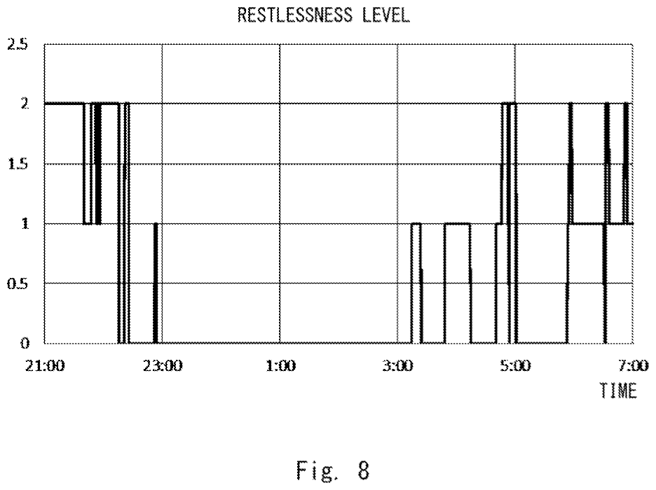

FIG. 8 shows the restlessness level calculated by the restlessness level determination unit 103. In FIG. 8, the horizontal axis indicates time, and the vertical axis indicates the restlessness level. In this example, it is assumed that the maximum restlessness level is level 2, and the minimum restlessness level is level 0. The level 2 indicates that the degree of the restless state is highest, or the possibility of being the restless state is highest. The level 0 indicates that the state of a patient is not the restless state (normal), or the possibility of being the restless state is lowest. The restlessness level determination unit 103 applies the movement quantity and the volume at each time, for example, to the identification model 210 (see FIG. 6), and calculates the restlessness level at each time.

The output unit (output means) 104 outputs the abnormality level determined by the abnormality level determination unit 103. The output unit 104 outputs the restlessness level calculated by the abnormality level determination unit 103 in chronological order, for example. The output unit 104 may give the restlessness level calculated by the abnormality level determination unit 103 as a label to sensor data of a sensor that measures the inner state included in the sensor group 130, and store it into a storage device or the like, which is not shown. Alternatively, when the restlessness level is equal to or higher than a threshold, the output unit 104 may notify a healthcare worker or the like that a patient is in the restless state. In this case, the output unit 104 may display that a patient is in the restless state on a display screen of a mobile information terminal device such as a smartphone or a tablet owned by a healthcare worker or the like, for example. Alternatively, the output unit 104 may notify that a patient is in the restless state by sound through earphones worn by a healthcare worker or the like. Further, the output unit 104 may display that a patient is in the restless state on a monitor installed in a nurse station or the like, or may notify that a patient is in the restless state by using a speaker installed in a nurse station or the like.

A threshold that serves as a criterion for the output unit 104 to give a notification may vary dynamically, not limited to a fixed value. The threshold can be determined on the basis of information indicating whether a patient is in abnormal condition or not, which is input by a healthcare worker through the input unit 150, for example. Given a notification that a patient is in the restless state, a healthcare worker goes to see the patient, and then inputs information indicating whether the patient is actually in the restless state or not to the abnormality determination apparatus 100. When the patient is not actually in the restless state, the output unit 104 may set the threshold to be greater than the current threshold. On the other hand, when there is no notification that a patient is in the restless state and the patient is actually in the restless state, the threshold is set to be smaller than the current threshold. The threshold may be changed after a healthcare worker inputs information a plurality of times.

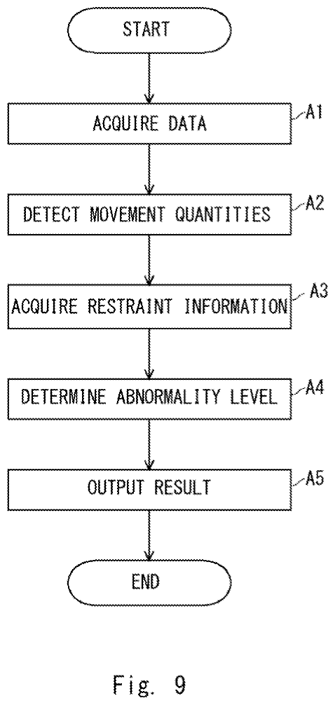

The operation procedure is described hereinbelow. FIG. 9 shows the operation procedure (abnormality determination method) in the abnormality determination apparatus 100. The movement amount detection unit 101 acquires image data and sensor data from the camera 120 and the sensor group 130, respectively (Step A1). Further, the volume detection unit 102 acquires audio data from the microphone 140. The movement amount detection unit 101 detects the movement quantities of a plurality of parts of a patient on the basis of the data acquired from the camera 120 and the sensor group 130 (Step A2). Further, the volume detection unit 102 detects the volume around the patient on the basis of the acquired audio data. When the volume is not used for the determination of the abnormality level, the acquisition of the audio data and the detection of the volume may be omissible.

The abnormality level determination unit 103 acquires the movement quantities of a plurality of parts detected by the movement amount detection unit 101, and the volume detected by the volume detection unit 102. Further, the abnormality level determination unit 103 acquires the restraint information input through the input unit 150 (Step A3). The abnormality level determination unit 103 determines the abnormality level on the basis of the movement quantities of a plurality of parts of the patient, the volume, and the restraint information (Step A4). In Step A4, the abnormality level determination unit 103 selects the identification model on the basis of the restraint information, for example, and applies the movement quantities of a plurality of parts of the patient and the volume to the selected identification model, and thereby calculates the abnormality level. The output unit 104 outputs the calculation result of the abnormality level (Step A5).

Prior to determining the abnormality level, the abnormality level determination unit 103 may determine whether the patient is conscious or unconscious. Whether the patient is conscious or not can be determined from the patient's action on the bed, eye movement and the like. When the patient is unconscious, the abnormality level determination unit 103 may determine that the patient is normal and set the value of the abnormality level to a value indicating “normal”. After that, the abnormality level determination unit 103 may wait for a predetermined period of time and then determine whether the patient is conscious or unconscious. When the abnormality level determination unit 103 determines that the patient is conscious, it may perform Step A4 and determine the abnormality level.

Note that, in a hospital, it is likely that a sheet or the like is put over a patient lying on a bed, which makes it difficult to determine a body part and motion only from image data. Further, in some cases, even when a sheet is not put over a patient, black and white image data that is captured by a near-infrared camera is output from the camera 120 in some cases when a light is out. This can make it difficult for the movement amount detection unit 101 to determine a body part, i.e., to specify the positions of the head, hands, and feet.

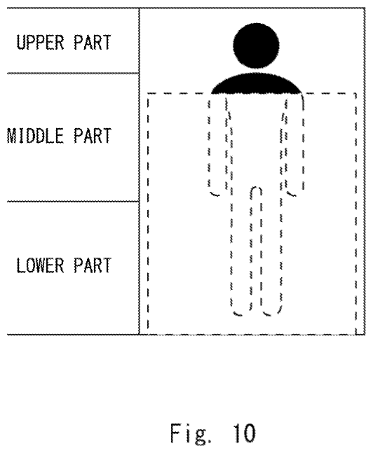

FIG. 10 shows a patient lying on a bed. In this example, a sheet is put over the patient, and the most part of the trunk and the limbs of the patient are hidden in the image data captured from directly above the bed. In such a case, the movement amount detection unit 101 may divide the area of the image data into a plurality of areas, and detect the movement quantity of a part of the patient corresponding to each area on the basis of the amount of change in the pixel value in each area. For example, the movement amount detection unit 101 divides the bed area into the upper part, the middle part, and the lower part, and calculates the amount of change in the pixel value in each part. To be specific, as shown in FIG. 10, the bed area is partitioned in proportions of 2:4:4 from above (from the head), for example, and those parts are assigned to the upper part, the middle part, and the lower part. The movement amount detection unit 101 may calculate the amount of change in the pixel value in each part, and detect the amount of change in the pixel as the movement quantity in each part. The proportion may be determined by using the ratio of the upper part, the middle part, and the lower part obtained when a sheet is not put over the patient.

In this example embodiment, the abnormality level determination unit 103 determines the abnormality level of a patient on the basis of the movement quantity, the volume, and the restraint information of the patient. The abnormality level determination unit 103 selects the identification model to be used for determining the abnormality level depending on the restraint information, for example, applies the movement quantity and the volume to the selected identification model, and thereby determines the abnormality level. In this way, even when the restraint status changes and thereby the correlation between the movement quantity and the abnormal condition changes, the abnormality level of the patient is determined accurately on the basis of the restraint status after change. The accuracy of detecting the abnormal condition of the patient is thereby improved in this example embodiment.

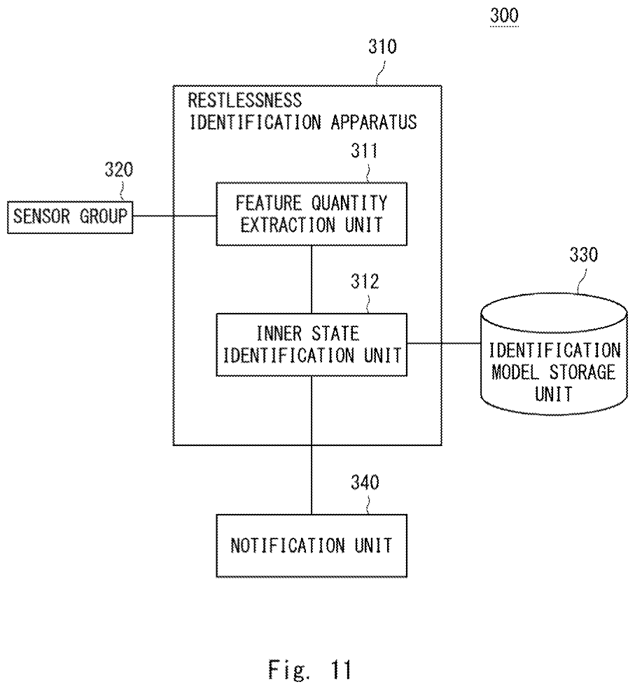

A second example embodiment of the present disclosure will be described hereinafter. In this example embodiment, a biological information processing system is provided. FIG. 11 shows a biological information processing system. A biological information processing system 300 includes a biological information processing apparatus (restlessness identification apparatus) 310, a sensor group 320, an identification model storage unit 330, and a notification unit 340. The restlessness identification apparatus 310 is configured as a computer device that includes a memory and a processor, for example. The identification model storage unit 330 is configured as a storage device such as HDD (Hard Disk Drive) or SSD (Solid State Drive), for example.

The sensor group 320 includes one or more sensors that acquire biological information (sensor data) of a monitored person such as a patient, for example. The sensor data contains data selected from a group including of heart rate, respiration rate, blood pressure, body temperature, consciousness, skin temperature, conductance skin response, electrocardiogram waveform, and electroencephalogram waveform.

The identification model storage unit 330 stores an identification model. The identification model is an identification model (identification parameter) for generating information indicating the level of the restless state on the basis of the sensor data obtained from the sensor group 320. The level of the restless state includes restless state, normal, and unclear, which is neither restless nor normal, for example. The restless state may be represented as a plurality of level values. For example, the restless state may be represented as three levels (highly restless, restless, and slightly restless). In this case, as the level is higher, the probability of taking problematic behavior is high, or the possibility of causing a severe problem is high. Unclear is a state where whether it is restless or normal is not easily determined. The identification model is generated by learning the relationship between the past sensor data and the past restless state or non-restless state, for example.

The biological information processing apparatus 310 includes a feature quantity extraction unit 311 and an inner state identification unit 312. The feature quantity extraction unit 311 acquires sensor data from the sensor group 320 and extracts a feature quantity from the acquired sensor data. As the feature quantity of a heart rate, for example, the feature quantity extraction unit extracts (calculates) at least one of the average in a given period, the variance (standard deviation), a difference between the average value and the current value, the derivative, the second order derivative, a specific frequency component in a given period, or the ratio of different frequency components. As the feature quantity of a temperature value, the feature quantity extraction unit 311 calculates at least one of the average in a given period, the variance (standard deviation), a difference between the average value and the current value, the derivative, or the second order derivative. The feature quantity extraction unit 311 outputs the extracted feature quantity in chronological order. The inner state identification unit 312 identifies the inner state (restless state) of a patient on the basis of the feature quantity extracted by the feature quantity extraction unit 311 and the identification model stored in the identification model storage unit 330. The inner state identification unit 312 outputs a score (restlessness score) indicating the level of the restless state, for example, as an identification result of the restless state.

The notification unit 340 notifies a healthcare worker or the like of the identification result of the restless state identified by the inner state identification unit 312. When the restlessness score output from the inner state identification unit 312 is equal to or higher than a predetermined value, for example, the notification unit 340 may notify a healthcare worker or the like that a patient is in the restless state. The notification unit 340 may include at least one of a lamp, an image display device, or a speaker, and notify a healthcare worker or the like that a patient is in the restless state by using at least one of light, image information, or sound. To be specific, the notification unit 340 may display a notification that a patient is in the restless state on a display screen of a mobile information terminal device such as a smartphone or a tablet owned by a healthcare worker or the like. Alternatively, the notification unit 340 may give a notification that a patient is in the restless state by sound through earphones worn by a healthcare worker or the like. Further, the notification unit 340 may display that a patient is in the restless state on a monitor installed in a nurse station or the like, or may notify that a patient is in the restless state by using a speaker installed in a nurse station or the like. The notification unit 340 notifies a healthcare worker of the restless state when a patient is experiencing restlessness before taking problematic behavior, which allows the healthcare worker or the like to care this patient before the patient takes problematic behavior.

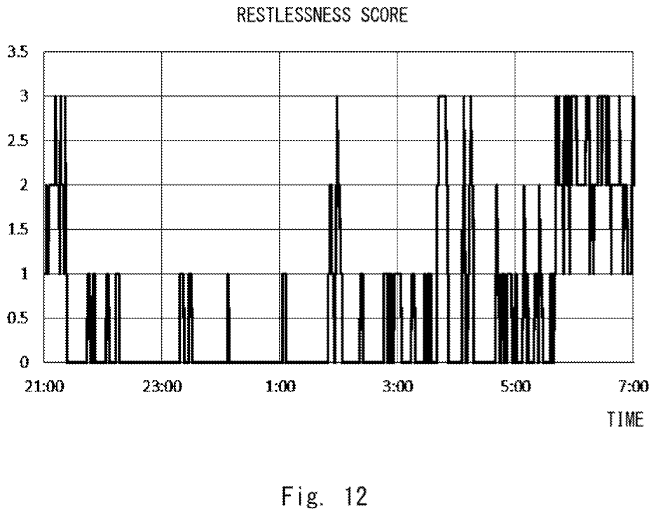

FIG. 12 shows a specific example of the restlessness score. In the graph shown in FIG. 12, the vertical axis indicates the restlessness score, and the horizontal axis indicates time. The graph in FIG. 12 shows the time variation of the restlessness score obtained as a result that the identification model is applied to the sensor data at a certain time in the inner state identification unit 312. In this example, the restlessness score takes a value from 0 to 3. It is assumed that the restlessness score “0” indicates that it is not the restless state (normal), or the possibility of being the restless state is lowest. It is also assumed that the restlessness score “3” indicates that the degree of the restless state is highest, or the possibility of being the restless state is highest. Such an identification model is generated by performing learning using training data where the label assigned in the normal state is “0” and the label assigned in the restless state is “3”.

The inner state identification unit 312 applies the feature quantity extracted from the sensor data that can vary from moment to moment to the identification model, and outputs the restlessness score in chronological order. The notification unit 340 notifies a healthcare worker or the like that a patient is in the restless state when the restlessness score is a predetermined value, such as 2 or higher, for example. A threshold that serves as a criterion to give a notification can be set as appropriate according to the identification model to use, other conditions and the like. The healthcare worker who has received the notification can go to see the condition of the patient. The healthcare worker may input information indicating that the patient is actually in the restless state or the patient is not actually in the restless state by using a terminal device such as a tablet placed bedside, for example. Further, the healthcare worker may input information about the details of care given to the patient, such as talking to the patient or adjusting the bed, by using a terminal device such as a tablet placed bedside, for example.

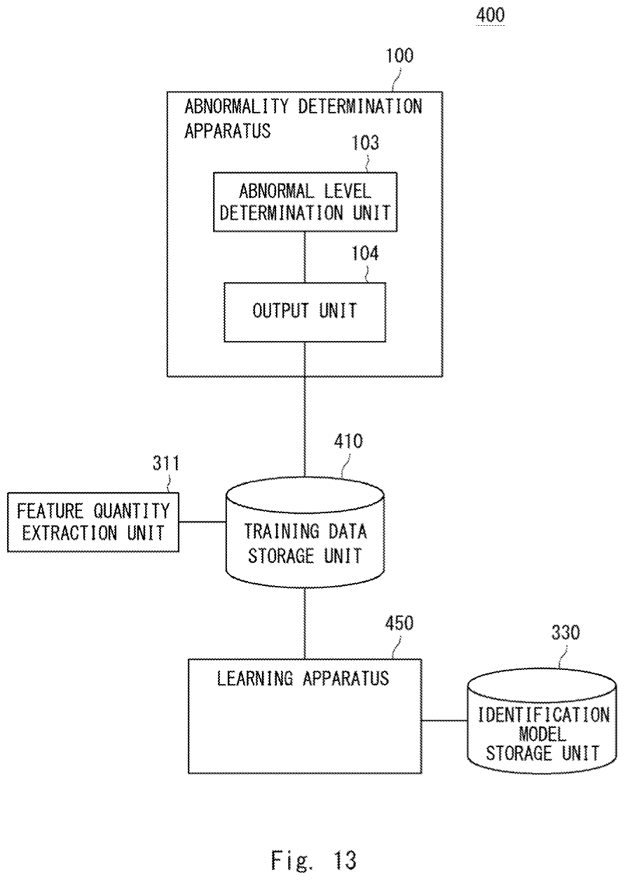

FIG. 13 shows a system used for learning of the identification model. This system 400 includes an abnormality determination apparatus 100, a feature quantity extraction unit 311, an identification model storage unit 330, a training data storage unit 410, and a learning apparatus 450. The abnormality determination apparatus 100 may be the same as the abnormality determination apparatus 100 shown in FIG. 2. In FIG. 13, the illustration of the movement amount detection unit 101, and the volume detection unit 102 and the like is omitted. The feature quantity extraction unit 311 and the identification model storage unit 330 may be the same as the feature quantity extraction unit 311 and the identification model storage unit 330 of the restlessness identification apparatus 310 shown in FIG. 11, respectively. The training data storage unit 410 is configured as a storage device such as HDD or SSD, for example.

The output unit 104 of the abnormality determination apparatus 100 outputs the abnormality level determined by the abnormality level determination unit 103 in chronological order. The training data storage unit 410 stores, as training data, the feature quantity extracted by the feature quantity extraction unit 311 and the abnormality level output from the abnormality determination apparatus 100. In the training data, the abnormality level output from the abnormality determination apparatus 100 is assigned as a label to the feature quantity extracted by the feature quantity extraction unit. The feature extracted by the feature quantity extraction unit 311 at each time and a determination result of the abnormality level at each time are accumulated as the training data in the training data storage unit 410.

The learning apparatus 450 generates an identification model for generating information indicating the level of the restless state on the basis of the sensor data obtained from the sensor group 320 (see FIG. 11) by supervised learning, for example, using the training data stored in the training data storage unit 410. The learning apparatus 450 stores the generated identification model into the identification model storage unit 330. The identification model stored in the identification model storage unit 330 is used for identification of the inner state in the restlessness identification apparatus 310 shown in FIG. 11.

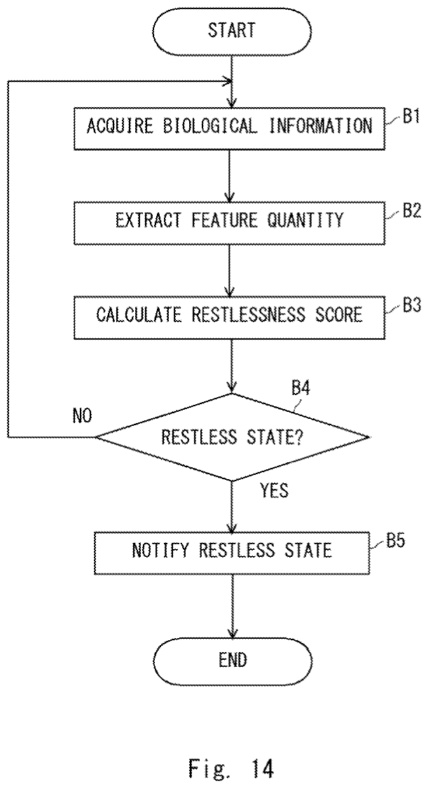

The operation procedure (biological information processing method) in the biological information processing system 300 is described hereinafter. FIG. 14 shows the operation procedure. The feature quantity extraction unit 311 of the biological information processing apparatus 310 acquires biological information (sensor data) from the sensor group 320 (Step B1). The feature quantity extraction unit 311 extracts the feature quantity from the acquired sensor data (Step B2). The inner state identification unit 312 applies the feature quantity extracted in Step B2 to the identification model stored in the identification model storage unit 330 and thereby calculates the restlessness score (Step B3).

The notification unit 340 determines whether a patient is in the restless state or not on the basis of the restlessness score calculated in Step B3 (Step B4). In Step B4, the notification unit 340 determines whether the restlessness score is a threshold or more, for example, and when it is a threshold or more, determines that the patient is in the restless state. When it is determined that the patient is not in the restless state in Step B4, the process returns to Step B1 and continues to acquire the biological information. When, on the other hand, it is determined that the patient is in the restless state in Step B4, the notification unit 340 notifies a healthcare worker or the like that the patient is in the restless state (Step B5).

In this example embodiment the restlessness level that is output from the abnormality determination apparatus 100 is used as the label of the training data. In the system 400, the feature extracted by the feature quantity extraction unit 311 and the restlessness level output from the abnormality determination apparatus 100 are accumulated as training data in the training data storage unit 410. By generating the identification model using the training data accumulated in this manner, the accuracy of identification results in the identification model that identifies the restless state of a patient from the biological information acquired using the sensor group 320 is expected to increase. When a patient is experiencing restlessness before taking problematic behavior, the notification unit 340 notifies a healthcare worker that the patient is in the restless state if it is determined using the identification model that the patient is in the restless state, and thereby the healthcare worker or the like can care this patient before the patient takes problematic behavior.

It is well known that the restless state of a patient includes quiet restlessness and intense restlessness. The quiet restlessness is an inner abnormality, and the intense restlessness is an abnormality in action. There is a correlation between the abnormal action level (action element) accompanying the restless state and the level of the inner restlessness (inner element). Specifically, a severe inner abnormality tends to cause abnormal action.

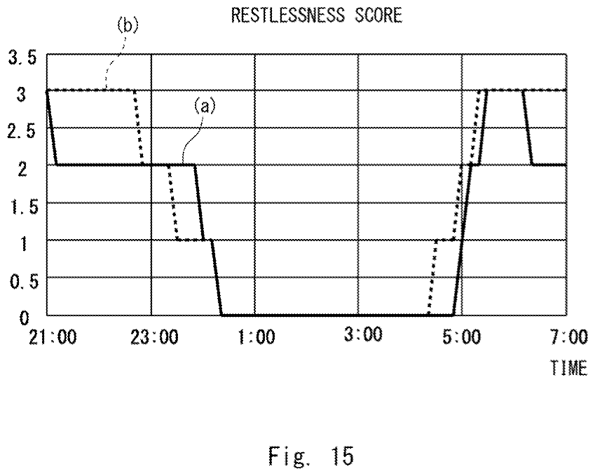

FIG. 15 shows a specific example of the action element and the inner element. In FIG. 15, the horizontal axis indicates time, and the vertical axis indicates the abnormal action level and the inner restlessness level. In FIG. 15, the graph (a) represents the action element, which is the abnormal action level accompanying the restless state. The graph (b) represents the inner element, which is the inner restlessness level. In this example embodiment, the restlessness level identified by the inner state identification unit 312 of the biological information processing apparatus 310 corresponds to the inner restlessness level. By detecting the inner element with use of the biological information processing apparatus 310, a healthcare worker or the like can detect the restless state before abnormal action occurs, and thereby carry out care before abnormal action occurs.

In each of the above example embodiments, the function of the abnormality determination apparatus 100, the function of each part of the biological information processing system 300, and the function of the learning apparatus 450 may be implemented using hardware or using software. Further, the function of the abnormality determination apparatus 100, the function of each part of the biological information processing system 300, and the function of the learning apparatus 450 may be implemented by combining hardware and software.

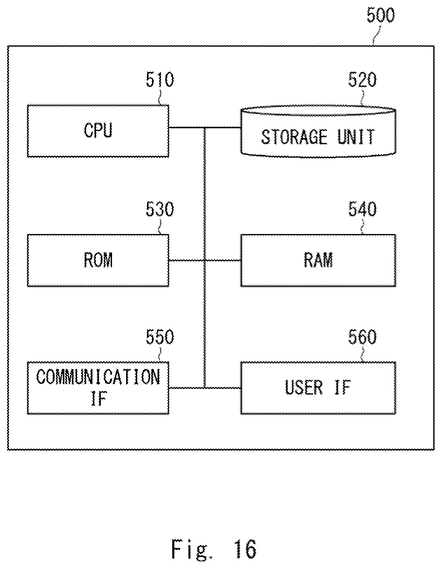

FIG. 16 shows a configuration example of an information processing device (computer device) that can be used for the abnormality determination apparatus 100, the restlessness identification apparatus 310, and the learning apparatus 450. An information processing device 500 includes a control unit (CPU: Central Processing Unit) 510, a storage unit 520, ROM (Read Only Memory) 530, RAM (Random Access Memory) 540, a communication interface (IF: Interface) 550, and a user interface 560.

The communication interface 550 is an interface for connecting the information processing device 500 and a communication network through a wired communication means, a wireless communication means or the like. The user interface 560 includes a display unit such as a display. The user interface 560 further includes an input unit such as a keyboard, a mouse, and a touch panel.

The storage unit 520 is an auxiliary storage device for storing various types of data. The storage unit 520 is not necessarily a part of the information processing device 500, and it may be an external storage device or a cloud storage that is connected to the information processing device 500 through a network. The ROM 530 is a nonvolatile storage device. A semiconductor storage device such as a flash memory with relatively small capacity can be used for the ROM 530, for example. A program executed by the CPU 510 can be stored in the storage unit 520 or the ROM 530.

The above-described program can be stored using any type of non-transitory computer readable media and provided to the information processing device 500. The non-transitory computer readable media include any type of tangible storage media. Examples of non-transitory computer readable media include magnetic storage media such as flexible disks, magnetic tapes or hard disks, optical magnetic storage media such as magneto-optical disks, optical disc media such as CD (Compact Disc) or DVD (Digital Versatile Disk), and semiconductor memories such as mask ROM, PROM (Programmable ROM), EPROM (Erasable PROM), flash ROM or RAM (Random Access Memory). The program may be provided to a computer using any type of transitory computer readable media. Examples of transitory computer readable media include electric signals, optical signals, and electromagnetic waves. Transitory computer readable media can provide the program to a computer via a wired communication line such as electric wires and optical fibers, or a wireless communication line.

The RAM 540 is a volatile storage device. A semiconductor memory device such as DRAM (Dynamic Random Access Memory) or SRAM (Static Random Access Memory) is used as the RAM 540. The RAM 540 can be used as an internal buffer that temporarily stores data or the like. The CPU 510 develops, on the RAM 540, a program stored in the storage unit 520 or the ROM 530 and executes it. The CPU 510 executes the program, and thereby the functions of at least some of the movement amount detection unit 101, the volume detection unit 102, the abnormality level determination unit 103, and the output unit 104 of the abnormality determination apparatus 100 shown in FIG. 2 are implemented. Further, the functions of at least some of the feature quantity extraction unit 311 and the inner state identification unit 312 of the restlessness identification apparatus 310 shown in FIG. 11 are implemented. Alternatively, the function of the learning apparatus 450 shown in FIG. 13 is implemented. The CPU 510 may include an internal buffer for temporarily storing data or the like.

While the present disclosure has been particularly shown and described with reference to example embodiments thereof, the present disclosure is not limited to the above-described example embodiments, and various changes and modifications may be made therein without departing from the spirit and scope of the present disclosure.

For example, the whole or part of the example embodiments disclosed above can be described as, but not limited to, the following supplementary notes.

### (Supplementary Note 1)

An abnormality determination apparatus comprising:

a movement quantity detection unit configured to detect movement quantities of a plurality of parts of a monitored person; and

an abnormality level determination unit configured to determine an abnormality level of the monitored person on the basis of the movement quantities of the plurality of parts and restraint information indicating a restraint status of the monitored person.

### (Supplementary Note 2)

The abnormality determination apparatus according to claim 1, wherein the restraint information contains information indicating a type of restraint used on the monitored person.

### (Supplementary Note 3)

The abnormality determination apparatus according to claim 2, wherein the restraint information further contains information related to a time period during which a type of restraint indicated by the information indicating a type of restraint is used on the monitored person.

### (Supplementary Note 4)

The abnormality determination apparatus according to any one of claims 1 to 3, further comprising:

a volume detection unit configured to detect a volume of at least one of a sound uttered by the monitored person or a sound generated due to behavior of the monitored person,

wherein the abnormality level determination unit determines the abnormality level on the basis of the movement quantities of the plurality of parts, the volume, and the restraint information indicating a restraint status of the monitored person.

### (Supplementary Note 5)

The abnormality determination apparatus according to any one of claims 1 to 4, wherein the movement quantity detection unit detects the movement quantities of the plurality of parts on the basis of image data obtained by capturing an image of the monitored person.

### (Supplementary Note 6)

The abnormality determination apparatus according to claim 5, wherein the movement quantity detection unit divides an area of the image data into a plurality of areas, and detects a movement quantity of a part of the monitored person corresponding to each area on the basis of an amount of change in a pixel value in each area.

### (Supplementary Note 7)

The abnormality determination apparatus according to any one of claims 1 to 6, wherein the movement quantity detection unit detects the movement quantity on the basis of sensor data acquired from a sensor group including one or more sensors.

### (Supplementary Note 8)

The abnormality determination apparatus according to any one of claims 1 to 7, wherein the abnormality level determination unit determines whether the monitored person is conscious or unconscious, and when the monitored person is unconscious, determines that the monitored person is normal.

### (Supplementary Note 9)

The abnormality determination apparatus according to any one of claims 1 to 8, wherein the abnormality level determination unit determines the abnormality level by selecting an identification model for calculating the abnormality level from the movement quantities of the plurality of parts on the basis of the restraint information, and applying the movement quantities of the plurality of parts to the selected identification model.

### (Supplementary Note 10)

The abnormality determination apparatus according to claim 9, wherein the abnormality level determination unit selects the identification model by referring to a table storing a type of restraint used on the monitored person and an identification model to be used for determination of the abnormality level.

### (Supplementary Note 11)

The abnormality determination apparatus according to claim 9 or 10, wherein the identification model is learned using training data containing the movement quantity and a label indicating a degree of abnormality of the monitored person.

### (Supplementary Note 12)

The abnormality determination apparatus according to any one of claims 1 to 11, further comprising:

an output unit configured to output biological information of the monitored person and the abnormality level in association with each other.

### (Supplementary Note 13)

An abnormality determination method comprising:

detecting movement quantities of a plurality of parts of a monitored person; and

determining an abnormality level of the monitored person on the basis of the movement quantities of the plurality of parts and restraint information indicating a restraint status of the monitored person.

### (Supplementary Note 14)

A computer readable medium storing a program causing a computer to execute a process comprising:

detecting movement quantities of a plurality of parts of a monitored person; and

determining an abnormality level of the monitored person on the basis of the movement quantities of the plurality of parts and restraint information indicating a restraint status of the monitored person.

### REFERENCE SIGNS LIST

10 ABNORMALITY DETERMINATION APPARATUS
11 MOVEMENT QUANTITY DETECTION MEANS
12 ABNORMALITY LEVEL DETERMINATION MEANS
30 RESTRAINT INFORMATION
100 ABNORMALITY DETERMINATION APPARATUS
101 MOVEMENT AMOUNT DETECTION UNIT
102 VOLUME DETECTION UNIT
103 ABNORMALITY LEVEL DETERMINATION UNIT
104 OUTPUT UNIT
120 CAMERA
130 SENSOR GROUP
140 MICROPHONE
150 INPUT UNIT
201 MOVEMENT QUANTITY
202 VOLUME
203 RESTRAINT INFORMATION
210 IDENTIFICATION MODEL
300 BIOLOGICAL INFORMATION PROCESSING SYSTEM
310 RESTLESSNESS IDENTIFICATION APPARATUS
311 FEATURE QUANTITY EXTRACTION UNIT
312 INNER STATE IDENTIFICATION UNIT
320 SENSOR GROUP
330 IDENTIFICATION MODEL STORAGE UNIT
340 NOTIFICATION UNIT
400 SYSTEM
410 TRAINING DATA STORAGE UNIT
450 LEARNING APPARATUS

What is claimed is:

1. An abnormality determination apparatus comprising:
a memory storing instructions, and
a processor configured to execute the instructions to implement:
a movement quantity detection unit configured to detect movement quantities of a plurality of parts of a monitored person; and
an abnormality level determination unit configured to determine an abnormality level of the monitored person on the basis of the movement quantities of the plurality of parts and restraint information indicating a restraint status of the monitored person.

2. The abnormality determination apparatus according to claim 1, wherein the restraint information contains information indicating a type of restraint used on the monitored person.

3. The abnormality determination apparatus according to claim 2, wherein the restraint information further contains information related to a time period during which a type of restraint indicated by the information indicating a type of restraint is used on the monitored person.

4. The abnormality determination apparatus according to claim 1,
wherein the processor further configured to execute the instructions to implement:
a volume detection unit configured to detect a volume of at least one of a sound uttered by the monitored person or a sound generated due to behavior of the monitored person, and
wherein the abnormality level determination unit determines the abnormality level on the basis of the movement quantities of the plurality of parts, the volume, and the restraint information indicating a restraint status of the monitored person.

5. The abnormality determination apparatus according to claim 1, wherein the movement quantity detection unit detects the movement quantities of the plurality of parts on the basis of image data obtained by capturing an image of the monitored person.

6. The abnormality determination apparatus according to claim 5, wherein the movement quantity detection unit divides an area of the image data into a plurality of areas, and detects a movement quantity of a part of the monitored person corresponding to each area on the basis of an amount of change in a pixel value in each area.

7. The abnormality determination apparatus according to claim 1, wherein the movement quantity detection unit detects the movement quantity on the basis of sensor data acquired from a sensor group including one or more sensors.

8. The abnormality determination apparatus according to claim 1, wherein the abnormality level determination unit determines whether the monitored person is conscious or unconscious, and when the monitored person is unconscious, determines that the monitored person is normal.

9. The abnormality determination apparatus according to claim 1, wherein the abnormality level determination unit determines the abnormality level by selecting an identification model for calculating the abnormality level from the movement quantities of the plurality of parts on the basis of the restraint information, and applying the movement quantities of the plurality of parts to the selected identification model.

10. The abnormality determination apparatus according to claim 9, wherein the abnormality level determination unit selects the identification model by referring to a table storing a type of restraint used on the monitored person and an identification model to be used for determination of the abnormality level.

11. The abnormality determination apparatus according to claim 9, wherein the identification model is learned using training data containing the movement quantity and a label indicating a degree of abnormality of the monitored person.

12. The abnormality determination apparatus according to claim 1, further comprising:
an output unit configured to output biological information of the monitored person and the abnormality level in association with each other.

13. An abnormality determination method comprising:
detecting movement quantities of a plurality of parts of a monitored person; and
determining an abnormality level of the monitored person on the basis of the movement quantities of the plurality of parts and restraint information indicating a restraint status of the monitored person.

14. A non-transitory computer readable medium storing a program causing a computer to execute a process comprising:
detecting movement quantities of a plurality of parts of a monitored person; and
determining an abnormality level of the monitored person on the basis of the movement quantities of the plurality of parts and restraint information indicating a restraint status of the monitored person.
